(19) United States
(12) Patent Application Publication
(10) Pub. No.: US 2025/0041315 A1
(43) Pub. Date: Feb. 6, 2025

(54) LEVONORGESTREL BUTANOATE FORMULATION AND METHODS RELATING THERETO

(71) Applicants: The USA, as Represented by the Secretary, Department of Health and Human Services, Bethesda, MD (US); SRI International, Menlo Park, CA (US)

(72) Inventors: Min Lee, San Diego, CA (US); Diana Blithe, Silver Spring, MD (US); Jia-Hwa Fang, Oakland, CA (US); Eduardo Ruiz, Newark, CA (US); Ken Chen, San Francisco, CA (US)

(73) Assignees: The USA, as Represented by the Secretary, Department of Health and Human Services, Bethesda, MD (US); SRI International, Menlo Park, CA (US)

(21) Appl. No.: 18/718,737

(22) PCT Filed: Dec. 13, 2022
(86) PCT No.: PCT/US2022/052704

§ 371 Date: Jun. 11, 2024

Related U.S. Application Data
(60) Provisional application No. 63/289,965, filed on Dec. 15, 2021.

(51) Int. Cl. A61K 31/565; A61K 9/16

(52) U.S. Cl. CPC A61K 31/565; A61K 9/1623; A61K 9/1652; A61K 9/1682

(57) ABSTRACT

Disclosed herein are formulation embodiments comprising levonorgestrel butanoate (“LNGB”) particles having particle sizes that facilitate administering a higher concentration of LNGB at lower volumes. The disclosed formulation embodiments exhibit long-lasting contraceptive effects and can be administered subcutaneously, which lends to their utility in acting as self-administrable contraceptive formulations that do not result in side effects associated with other contraceptive agents.

### CROSS REFERENCE TO RELATED APPLICATION

This application claims the benefit of and priority to the earlier filing date of U.S. Provisional Patent Application No. 63/289,965, filed on Dec. 15, 2021, the entirety of which is incorporated herein by reference.

### ACKNOWLEDGMENT OF GOVERNMENT SUPPORT

This invention was made with government support under project number ZHD008981-01, awarded by the National Institutes of Health, Eunice Kennedy Shriver National Institute of Child Health and Human Development. The government has certain rights in the invention.

### FIELD

The present disclosure is directed to embodiments of a levonorgestrel butanoate formulation and methods of making and using the same.

### BACKGROUND

Long-acting contraceptive methods, such as intrauterine devices (IUD), have a typical failure rate of ˜1%. Methods that require daily action such as Combination Oral Contraceptives (COC) or Progestin Only Pills (POP) have a typical failure rate of 7% due to user error. The typical failure rate of Depomedroxyprogesterone Acetate (DMPA), the only injectable product in the U.S., is 4%. The DMPA regimen calls for injections at 3-month intervals. A longer duration of action may improve the typical rate of effectiveness by requiring fewer injections per year. Concerns have been raised about possible increased risk of HIV acquisition with use of DMPA which may be attributable to glucocorticoid activity of medroxyprogesterone acetate. And, DMPA is associated with weight gain and may cause a decrease in bone mineral density, prompting the FDA to require a black-box warning regarding possible impact on bone fracture risk with long term use or effects in women younger than 18. Injectable methods provide several benefits over other methods, thus, there is a need for additional injectable contraceptive options that are safe and effective.

Obesity is a major public health issue facing the United States population and is an independent risk for venous thromboembolism (VTE); therefore, there is a public health need to develop effective contraception for obese woman that does not increase the risk of VTE. Most conventional birth control pills contain a synthetic estrogen, ethinyl estradiol (EE), that is associated with an increased risk of VTE. The combination of obesity and EE further increases VTE risk. Women with health conditions, such as moderate or morbid obesity, hypertension, and diabetes, may be advised to avoid use of EE-containing contraceptives, but their risk of VTE due to pregnancy is markedly increased over the risks associated with use of EE-containing products. Although recently introduced combination birth control pills with estradiol may have slightly lower risk of VTE as compared to those containing EE, the benefit is not clearly established, and they are underutilized due to poor tolerance and cycle control.

Levonorgestrel butanoate (“LNGB”) is a steroidal progestin that was in development by the World Health Organization (WHO) in collaboration with institutions around the world including the National Institute of Child Health and Human Development as a long-acting injectable contraceptive since the early 1980s. The first clinical study was conducted at two World Health Organization Collaborating Centers for Research in Human Reproduction, located in Mexico City and London. Conventional LNGB formulations, such as the WHO formulation, however, exhibit undesirable stability issues, which reduces contraceptive activity and the ability to manufacture at large scale.

Developing an effective contraceptive product that is safer for women with high-risk conditions and that exhibits sufficient long-lasting contraceptive effects is important public health need.

### SUMMARY

Disclosed herein are embodiments of a pharmaceutical formulation, comprising: levonorgestrel butanoate particles; a wetting agent; and a dispersing agent, an emulsifier, or a combination thereof. Unique combinations of particle size and concentration of the levonorgestrel butanoate are described.

Also disclosed are embodiments of a method of making a pharmaceutical formulation according to the present disclosure, the method comprising: subjecting levonorgestrel butanoate to sterile filtration to provide a filtrate comprising sterile levonorgestrel butanoate; producing levonorgestrel butanoate particles from the filtrate; and combining the levonorgestrel butanoate particles with one or more of the wetting agent, the dispersing agent, the emulsifier, or the combination thereof; wherein the method does not comprise using irradiation for any sterilization.

Also disclosed are embodiments of a method of making a pharmaceutical formulation according to the present disclosure, the method comprising: micronizing levonorgestrel butanoate to provide the levonorgestrel butanoate particles and combining the levonorgestrel butanoate particles with one or more of the wetting agent, the dispersing agent, the emulsifier, or the combination thereof; wherein the method does not comprise using irradiation for any sterilization.

Also disclosed are embodiments of a method of providing contraception to a subject, comprising administering a dosage of levonorgestrel butanoate ranging from 20 mg to 140 mg by administering a formulation embodiment as described herein to the subject via injection.

Also disclosed are embodiments of a method of treating or preventing a progestin-related disease or condition in a subject, comprising administering a formulation embodiment as described herein to the subject.

The foregoing and other objects and features of the present disclosure will become more apparent from the following detailed description, which proceeds with reference to the accompanying figures.

### BRIEF DESCRIPTION OF THE DRAWINGS

FIGS. 1A and 1B are photographic images showing levonorgestrel butanoate (“LNGB”) particles before (FIG. 1A) and after (FIG. 1B) being exposed to an irradiation-based sterilization step and which shows that irradiation causes the LNGB to have an undesirable yellow color.

FIG. 2 is a High-Performance Liquid Chromatography (HPLC) trace obtained from analyzing LNGB particles prior to any irradiation-based sterilization step.

FIG. 3 is an HPLC trace obtained from analyzing LNGB particles after an irradiation-based sterilization step.

FIG. 4 is an HPLC trace obtained from analyzing LNGB particles after an irradiation-based sterilization step, wherein the sample was purged with N₂.

FIG. 5 is an HPLC trace obtained from analyzing LNGB particles after an irradiation-based sterilization step, wherein the sample was purged with Ar.

FIG. 6 is a graph showing “return to ovulation” results obtained from injecting an exemplary LNGB formulation of the present disclosure different administration routes [(i) intramuscular (“IM”) injection; and (ii) subcutaneous (“SC”) injection] and different concentrations [(i) 70 mg/mL; and (ii) 20 mg/mL).

### DETAILED DESCRIPTION

### Overview of Terms

The following explanations of terms and/or symbols are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting. Any functional group disclosed herein can be substituted or unsubstituted, unless otherwise indicated herein.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g. around an amide bond or between two directly attached rings such as pyridinyl rings, biphenyl groups, and the like, atropisomers are also possible and are also specifically included in the compounds disclosed herein.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

To facilitate review of the various embodiments of the disclosure, the following explanations of specific terms are provided.

Administer (or Administering or Administration): To expose a subject to a formulation embodiment of the present disclosure, including any components thereof. Modes of administration are discussed herein and can include, but are not limited to, injection (such as intravenous, intraperitoneal, subcutaneous, intramuscular, or intrathecal), topical, ocular, oral, buccal, systemic, nasal, transdermal (e.g., by mixing with a penetrating agent, such as DMSO), rectal, vaginal, a form suitable for administration by inhalation or insufflation, a form suitable for implantation, or any combination thereof. Administration as used herein also contemplates self-administration wherein the exposed subject carries out the administration.

Pharmaceutically Acceptable Excipient: A substance, other than the active ingredient (such as LNGB), that is included in a formulation of LNGB. As used herein, an excipient may be incorporated within particles of LNGB, or it may be physically mixed with particles of LNGB. An excipient also can be in the form of a solution, suspension, emulsion, or the like. An excipient can be used, for example, to dilute an active ingredient and/or to modify properties of a pharmaceutical composition. Excipients can include, but are not limited to, wetting agents, dispersing agents, thickening agents, antiadherents, binders, coatings, enteric coatings, disintegrants, flavorings, sweeteners, colorants, lubricants, glidants, sorbents, preservatives, buffers, emulsifiers, adjuvants, carriers, vehicles, isotonicity agents, fillers, pharmaceutically acceptable solvents, stabilizers, flocculating agents, suspension agents, electrolytes, antioxidants, chelating agents, or any combination thereof. Exemplary excipients are described herein. Further additional exemplary excipients may include may be magnesium stearate, stearic acid, vegetable stearin, sucrose, lactose, starches, xylitol, sorbitol, maltitol, polyethylene glycol (PEG), tocopheryl polyethylene glycol 1000 succinate (also known as vitamin E TPGS, or TPGS), dipalmitoyl phosphatidyl choline (DPPC), vitamin A, vitamin E, vitamin C, retinyl palmitate, selenium, cysteine, methionine, citric acid, sodium citrate, sugar, silica, talc, magnesium carbonate, sodium starch glycolate, tartrazine, aspartame, sesame oil, propyl gallate, sodium metabisulphite, lanolin, or any combination thereof. If water is included as a pharmaceutically acceptable excipient, it typically is sterile water for injection (“WFI”), USP grade.

Pharmaceutically Acceptable Salt: Pharmaceutically acceptable salts of LNGB and/or other compounds and/or formulation components described herein that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the formulation components form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

Prodrug: Compound embodiments disclosed herein, such as LNGB, that are transformed, most typically in vivo, to yield a biologically active compound, particularly the parent compound (e.g., LNG), for example, by enzymatic hydrolysis in the gastrointestinal (GI) tract (including the stomach and gut). Common examples of prodrug moieties include, but are not limited to, pharmaceutically acceptable ester, carbonate, amide, and/or carbamate forms of a compound having an active form bearing a carboxylic acid moiety and/or a hydroxyl group. Examples of pharmaceutically acceptable esters of the compound embodiments of the present disclosure include, but are not limited to, esters of phosphate groups and carboxylic acids, such as aliphatic esters, particularly alkyl esters (for example C₁₋₆ alkyl esters, such as butanoate groups). Other prodrug moieties include phosphate esters, such as —CH₂—O—P(O)(OR^{a})₂ or a salt thereof, wherein R^{a} is hydrogen or aliphatic (e.g., C₁₋₆alkyl). Acceptable esters also include cycloalkyl esters and arylalkyl esters such as, but not limited to, benzyl. Examples of pharmaceutically acceptable amides of the compound embodiments of this disclosure include, but are not limited to, primary amides, and secondary and tertiary alkyl amides (for example with between one and six carbons). Amides and esters of disclosed exemplary embodiments of compound embodiments according to the present disclosure can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

Subject: Mammals and other animals, such as humans, companion animals (e.g., dogs, cats, rabbits, etc.), utility animals, and feed animals; thus, disclosed methods are applicable to both human therapy and veterinary applications.

Symptom (or Sign): Any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to, any measurable parameters such as tests for detecting a neurodegenerative disorder or disease.

Additional Therapeutic Agent: An optional component of certain formulation embodiments disclosed herein that are included in addition to any LNGB. In some embodiments, the additional therapeutic agent is a biologically active compound. Exemplary, but non-limiting, additional therapeutic agents can include gonadotropin-releasing hormone (“GnRH”) antagonists and/or agonists, E3 ubiquitin ligase recruiting ligands, anticancer agents (e.g., chemotherapy agents), kinase antagonists and/or agonists, GPCR antagonists and/or agonists, antimalarial agents, antifungal agents, antiviral agents, antibacterial agents, immunosuppressants, anti-inflammatory agents, pulmonary agents, and the like.

Therapeutically Effective Amount: An amount of LNGB, or a formulation thereof, sufficient to provide or cause a contraceptive effect (e.g., inhibit ovulation) in a subject, and in particular embodiments, a female subject; and/or treat a specified disorder or disease, or to ameliorate or eradicate one or more of its symptoms, and/or to prevent the occurrence of the disease or disorder.

Treating/Treatment: Treatment of a disease or condition of interest in a land mammal, including but not limited to, a human, canine, or feline, having the disease or condition of interest, and includes by way of example, and without limitation:
(i) prophylactic administration to prevent the disease or condition from occurring in a subject, or to ameliorate symptoms associated with the condition if required in particular, when such subject is predisposed to the condition but has not yet been diagnosed as having it;
(ii) inhibiting the disease or condition, for example, arresting or slowing its development;
(iii) relieving the disease or condition, for example, causing regression of the disease or condition or a symptom thereof; or
(iv) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### INTRODUCTION

Levonorgestrel butanoate (“LNGB”), also referred to in the art as levonorgestrel 17ß-butanoate, is a butyl prodrug of levonorgestrel (LNG), a widely use progestin. LNGB is a long-acting injectable means for contraception. As a progestin, the mode of LNG's contraceptive effect is through inhibiting ovulation and potentially additional protective mechanism as cervical mucus may have a role. LNGB is a long-acting injectable compound that can be used without increase in veinous thrombosis embolism (VTE) risk. Additionally, injections of LNGB improves compliance over progestin-only daily pills as they can be carried out via self-administration subcutaneously. Such flexibility in administration would greatly benefit the user as one does not have to go into doctor's office for a single injection that takes less than a minute. Further, levonorgestrel is not glucocorticoid receptor active, which is a receptor that is thought to influence weight gain.

A LNGB-containing contraceptive composition has been developed by the World Health Organization (WHO); however, this composition exhibits aggregation of LNGB particles, resulting in loss of product stability and reproducibility of clinical batches. Other compositions have been developed that attempted to address this aggregation/stability issue; however, one such composition resulted in a shorter than anticipated duration of activity. This formulation was similar in content to the WHO formulation, but included a stabilizing excipient to prevent the aggregation that was observed with WHO formulation. Other compositions in the art exhibit impurity issues that prevent their use in FDA clinical trials and thus do not present viable options for commercial formulations.

Improved formulations of LNGB are needed in the art as LNG is a progestin that is established to be one of the safest and most effective agents in use for contraception. Further, women with health conditions, such as moderate or morbid obesity, hypertension and diabetes may be advised to avoid use of EE-containing contraceptives, but their risk of VTE due to pregnancy is markedly increased over the risks associated with use of EE-containing products. As indicated above, LNGB would not increase the risk of VTE. Also needed are formulations that can be administered in a manner that facilitates concealability of the administration method. For example, an injectable method has the benefit of privacy as the user retains no evidence of use following the injection. And, injectable methods are relatively long-acting and have a lower typical failure rate than combined hormonal birth control pills, patches or progestin only pills (“POPs”). POPs require strict adherence to taking the POP at the same time every day (or not greater than a 12-hour difference, as with the desogestrel POP), and effectiveness may be reduced in obese women.

Described herein are improved formulation embodiments comprising LNGB that exhibit desirable activity and that can be used to administer higher concentrations of LNGB to provide longer lasting contraceptive effects with less volume needed for administration. Additionally, the disclosed formulations are suitable for subcutaneous administration and thus are user friendly as they can be self-administered. And, the disclosed formulation embodiments exhibit excellent stability of the LNGB particles.

### Formulation Embodiments

Disclosed herein are embodiments of a formulation comprising levonorgestrel butanoate (“LNGB”) and one or more pharmaceutically acceptable excipients. In particular embodiments, the formulation is an aqueous-based suspension of particles of LNGB in combination with the one or more pharmaceutically acceptable excipients. The particles of LNGB are sized-controlled and exhibit high stability without aggregation and/or degradation. In particular embodiments, the LNGB particles of the formulation embodiments exhibit particle sizes that facilitate the ability to increase LNGB concentration in the formulations.

In particular embodiments, the formulation comprises the LNGB at a concentration greater than 15 mg/mL, such as 20 mg/mL to 100 mg/mL, or greater than 20 mg/mL to less than 100 mg/mL, or greater than 20 mg/mL to 90 mg/mL, or 20 mg/mL to 90 mg/mL, or 30 mg/mL to 90 mg/mL, or 40 mg/mL to 90 mg/mL, 50 mg/mL to 90 mg/mL, or 60 mg/mL to 90 mg/mL, or 70 mg/mL to 90 mg/mL, or 80 mg/mL to 90 mg/mL. In particular embodiments, the LNGB concentration ranges from 30 mg/mL to less than 90 mg/mL, or greater than 30 mg/mL to less than 80 mg/mL, or 40 mg/mL to 80 mg/mL, 50 mg/mL to 80 mg/mL, or 60 mg/mL to 80 mg/mL, or 70 mg/mL to 80 mg/mL. In particular embodiments, the LNGB is included at a concentration ranging from 30 mg/mL to 100 mg/mL such as 20 mg/ml, 30 mg/mL, 40 mg/mL, 50 mg/mL, 60 mg/mL, 70 mg/mL, 80 mg/mL, 90 mg/mL, or 100 mg/mL.

In some embodiments, the amount of LNGB included in the formulation can range from greater than 2% (w/v) based on the total weight or volume of the formulation, such as 3% to 15% (w/v), or 4% to 15% (w/v), or 5% to 15% (w/v), or 6% to 15% (w/v), or 7% to 15% (w/v), or 8% to 15% (w/v), or 9% to 15% (w/v), or 10% to 15% (w/v), or 11% to 15% (w/v), or 12% to 15% (w/v), or 13% to 15% (w/v), or 14% to 15% (w/v). In particular embodiments, the amount of LNGB included in the formulation is 2% (w/v), 5% (w/v), 7% (w/v), 8% (w/v), 10% (w/v), or 15% (w/v).

The formulation embodiments can be formulated so as to deliver the LNGB in dosage amounts ranging from 20 mg to 160 mg, such as 30 mg to 140 mg, or 30 mg to 120 mg, or 30 mg to 100 mg, or 30 mg to 80 mg. In some embodiments, the dosage amount can range from 30 mg to 80 mg, or 40 mg to 80 mg, or 50 mg to 80 mg, or 60 mg to 80 mg, or 70 mg to 80 mg. In particular embodiments, the dosage amount is 30 mg, 40 mg, 50 mg, 60 mg, 70 mg, 80 mg, 100 mg, 120 mg, 140 mg, or 150 mg. In representative embodiments, a dosage of 40 mg at an LNGB concentration of 20 mg/mL, or 40 mg at an LNGB concentration of 70 mg/mL, or 50 mg at an LNGB concentration of 70 mg/mL, or 60 mg at an LNGB concentration of 70 mg/mL, or 70 mg at an LNGB concentration of 70 mg/mL is administered.

The LNGB particles included in the formulation can have median (D₅₀) particle sizes ranging from 5 μm to 20 μm, such as 7 μm to 16 μm, or 9 μm to 16 μm, or 11 μm to 16 μm, or 12 μm to 16 μm, or 13 μm to 16 μm. In exemplary embodiments, the LNGB particles have median particle sizes ranging from 11 μm to 15 μm, such as 12 μm to 15 μm, or 12.6 μm to 15 μm, or 12 μm to 14 μm, including 11.3 μm, 12.4 μm, 13.2 μm, 13.7 μm, or 14.2 μm. In independent embodiments, the median particle size is not, or is other than, 6.3 μm, 6.4 μm, 6.6 μm, 23.1 μm, 22.7 μm, 18.6 μm, 18.0 μm, 18.4 μm, 21.0 μm, or 15.8 μm. In exemplary embodiments, the LNGB particles have a median particle size of 11 μm to 13 μm, such as 11.3 μm or 12.4 μm; or a median particle size of 13 μm to 15 μm, such as 13 μm to 14 μm, or 13.2 μm, 13.7 μm, or 14.2 μm. In some such embodiments, the LNGB particles can be included in the formulation at concentrations ranging from 20 mg/mL to 100 mg/mL, such as 20 mg/mL, 30 mg/mL, 40 mg/mL, 50 mg/mL, 60 mg/mL, 70 mg/mL, 80 mg/mL, 90 mg/mL, or 100 mg/mL.

The formulation further includes one or more pharmaceutically acceptable excipients. Such pharmaceutically acceptable excipients can include, but are not limited to, wetting agents, dispersing agents, emulsifiers, thickening agents, buffers, and/or preservatives. In particular embodiments, the pharmaceutically acceptable excipients include at least one wetting agent, at least one dispersing agent, at least one thickening agent, at least one buffer component, and at least one preservative. In other embodiments, the pharmaceutically acceptable excipients include at least one dispersing agent, at least one thickening agent, at least one buffer component, and at least one preservative. In exemplary embodiments, the pharmaceutically acceptable excipients include a wetting agent, a dispersing agent, a thickening agent, a preservative, a buffer component comprising two buffers, and an optional emulsifier. In yet additional exemplary embodiments, the pharmaceutically acceptable excipients include a wetting agent, two dispersing agents, a thickening agent, a preservative, and a buffer component comprising two buffers. In yet additional exemplary embodiments, the pharmaceutically acceptable excipients include two dispersing agents or a dispersing agent and an emulsifier; a thickening agent; a preservative; and a buffer component comprising two buffers. In some embodiments, the pharmaceutically acceptable excipients can further include an isotonicity agent, a filler, a solvent, a stabilizer, a lubricant, a flocculating agent, a suspension agent, an electrolyte, an antioxidant, a chelating agent, or any combination thereof.

Wetting agents and dispersing agents that can be used in the disclosed formulation embodiments can include, but are not limited to, sorbitan surfactants, such as polysorbate surfactants (e.g., polysorbate 20, polysorbate 40, polysorbate 60, or polysorbate 80); sorbitan fatty acid esters (e.g., sorbitan monolaurate, sorbitan monopalmitate, sorbitan monostearate, sorbitan monooleate, sorbitan tristearate, sorbitan sesquioleate, sorbitan trioleate, or combinations thereof); polyalkylene oxide-containing compounds (e.g., polyoxyalkylated fats, polyoxyalkylated glycerides, polyoxyalkylated fatty acids, and the like); alkylphenolic compounds; sulfate surfactants (e.g., sodium lauryl sulfate and the like); glycerylmonooleate; lecithin; methyl laurate; linolizated glycerides (e.g., linolizated oleotriglycerides and the like); and combinations thereof. In particular embodiments, the wetting agent is a sorbitan surfactant, such as a polysorbate surfactant (e.g., polysorbate 20, polysorbate 40, polysorbate 60, or polysorbate 80). In particular embodiments, the dispersing agent is a sorbitan fatty acid ester, such as sorbitan monolaurate, sorbitan monopalmitate, sorbitan monostearate, sorbitan monooleate, sorbitan tristearate, sorbitan sesquioleate, sorbitan trioleate; a sulfate surfactant (e.g., sodium lauryl sulfate); or combinations thereof. In representative embodiments, a combination of polysorbate 80 (TWEEN® 80) and sorbitan monopalmitate (Span® 40) is used. In other representative embodiments, a combination of polysorbate 80 (TWEEN® 80), sorbitan monopalmitate (Span® 40), and sorbitan monolaurate (Span® 20) is used. In yet additional embodiments, a combination of sorbitan monopalmitate (Span® 40) and sodium lauryl sulfate is used. In yet additional embodiments, sorbitan monolaurate (Span® 20) is used. The total amount of the wetting agent and/or dispersing agent that can be used in the formulation can range from 0.1% w/v to 11% w/v, such as 0.2% w/v to 10.5% w/v, or 0.4% w/v to 10.5% w/v, or 0.9% w/v to 10.5% w/v, or 6% w/v to 10.5% w/v, or 7% w/v to 10.5% w/v, or 10% w/v to 10.5% w/v. In particular embodiments, the total amount of the wetting agent and/or dispersing agent used is 0.2%, 0.4%, 0.9%, 6.2%, 7.2%, or 10.2% w/v. In any or all of these embodiments, the total amount of wetting agent can be provided by using a single wetting agent or a combination of a wetting agent and a dispersing agent, or a combination of two or more dispersing agents. In some embodiments where a wetting agent and a dispersing agent are used, such as polysorbate 80 (TWEEN® 80) and sorbitan monopalmitate (Span® 40), the ratio of the wetting agent to dispersing agent can range from 2:0.1 to 6:0.1, such as 3:0.1 to 5:0.1 (polysorbate 80: sorbitan monopalmitate). In an independent embodiment, if polysorbate 80 and sorbitan monopalmitate are used in the formulation, they are included at a ratio that is not, or is other than, 1:1. In particular exemplary embodiments, polysorbate 80 is used in an amount ranging from 0.1% to 10% w/v, such as 0.2% to 10% w/v, or 6% to 10% w/v, or 6%, 7%, 8%, 9%, or 10% w/v. In additional exemplary embodiments, sorbitan monopalmitate is used in an amount ranging from 0.05% to 0.2% w/v, such as 0.1% to 0.2% w/v. In additional exemplary embodiments, sorbitan monolaurate is used in an amount ranging from 0.0.5% to 0.2% w/v, such as 0.1% to 0.2% w/v. In additional exemplary embodiments, sodium lauryl sulfate is used in an amount ranging from greater than 0% to 0.7% w/v, such as 0.1% to 0.7% w/v.

Thickening agents that can be used in the disclosed formulation embodiments can include, but are not limited to, cellulose-based materials, such as carboxymethyl cellulose (or salt forms thereof, including sodium or calcium carboxymethyl cellulose), methyl cellulose, hydroxypropylcellulose, hydroxyethylcellulose, hydroxypropylmethylcellulose, and/or crosslinked forms thereof; polyvinyl alcohol; polyvinylpyrrolidone; acacia; gelatin; or combinations thereof. In particular embodiments, the thickening agent is a carboxymethyl cellulose compound, such as sodium carboxymethylcellulose. The amount of thickening agent that can be used in disclosed formulation embodiments can range from 0.4% to 2% w/v, such as 0.5% to 2% w/v, or 1% w/v to 2% w/v, or 0.5%, 0.6%, 0.7%, 0.8%, 0.9%, or 1% w/v.

Emulsifiers that can be used in the disclosed formulation embodiments can include, but are not limited to, ionic or non-ionic surfactants. In some embodiments, the emulsifier is a non-ionic polyalkylene oxide-based surfactants, such as polyalkoxylated castor oil compounds (e.g., polyoxyl-35 castor oil [or “Kolliphor®EL”]) and/or polyalkylene oxide hydroxylated fatty acid (e.g., polyethylene glycol (15)-hydroxystearate [“Kolliphor®HS”]); or combinations thereof. The amount of emulsifier that can be included in disclosed formulation embodiments can range from greater than 0% to 8% w/v, such as 2% to 7.5% w/v, or 3% to 7 w % w/v, or 4% to 7% w/v, or 4.4% to 7%, or 5% to 7% w/v, or 3%, 4%, 4.4%, 5%, 6%, or 7% w/v.

Preservatives that can be used in the disclosed formulation embodiments include, but are not limited to, paraben compounds, such as methyl paraben, propyl paraben, butyl paraben, and the like; phenylmercuric compounds, such as phenylmercuric acetate, phenylmercuric borate, phenylmercuric nitrate, and the like; benzyl compounds, such as benzyl alcohol, benzalkonium chloride, and the like; aromatic compounds, such as thiomerosal, phenol, m-cresol, and the like; and alkyl halides, such as chlorobutanol. In exemplary embodiments, the preservative is benzyl alcohol. The amount of preservative that can be used in disclosed formulation embodiments can range from greater than 0% to 5% w/v, such 0.5% to 2% w/v, or 1% to 2% w/v, or 1.03% w/V.

Buffer components that can be used in formulation embodiments disclosed herein can include, but are not limited to, phosphate buffers, such as sodium phosphate dibasic, sodium dihydrogen phosphate dihydrate, or the like; acetate buffers, such as sodium acetate, tris-acetate, triethylammonium acetate, and the like; citrate buffers, such as sodium citrate and the like; tartrate buffers, such as sodium tartrate dibasic dihydrate, ammonium tartrate dibasic, potassium sodium tartrate, potassium sodium tartrate tetrahydrate, and the like; and amine-based buffers, such as triethanolamine and the like; or combinations thereof. In exemplary embodiments, the buffer component includes a combination of two buffers, such as sodium phosphate dibasic and sodium dihydrogen phosphate dihydrate. The total amount of the buffer component that can be used in disclosed formulation embodiments can range from greater than 0% to 2% w/v, such as 0.2% to 2% w/v, or 1% to 2% w/v, or 1.5% to 2% w/v, or 0.3%, 1.49%, or 1.79% w/v.

The formulation embodiments further comprise a balance of sterile water, such as sterile water suitable for injection (e.g., sterile WFI USP), to arrive at the final volume of the formation, such as 100%.

In particular embodiments, the formulation comprises, consists essentially of, or consists of a combination of LNGB having a median particle size ranging from 10 μm to 16 μm, such as 13.2 μm, 13.7 μm, or 15.4 μm; a wetting agent, such as polysorbate 80; a dispersing agent, such as sorbitan monopalmitate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In particular embodiments, the formulation comprises, consists essentially of, or consists of a combination of LNGB; a wetting agent, such as polysorbate 80; a dispersing agent, such as sorbitan monolaurate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In yet additional embodiments, the formulation comprises, consists essentially of, or consists of a combination of LNGB having a median particle size ranging from 10 μm to 17 μm, such as 11.6 μm, 13.6 μm, 14.2 μm, or 16.3 μm; two dispersing agents, such as sodium lauryl sulfate and sorbitan monopalmitate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In yet further embodiments, the formulation comprises, consists essentially of, or consists of a combination of LNGB; a wetting agent, such as polysorbate 80; two dispersing agents, such as sorbitan monopalmitate and sorbitan monolaurate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In yet further embodiments, the formulation comprises, consists essentially of, or consists of a combination of LNGB having a median particle size ranging from 10 μm to 15 μm, such as 11.3 μm or 14.2 μm; a dispersing agent, such as sorbitan monopalmitate; an emulsifier, such as polyoxyl-35 castor oil or polyethylene glycol (15)-hydroxystearate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In yet further embodiments, the formulation comprises, consists essentially of, or consists of a combination of LNGB having a median particle size ranging from 11 μm to 17 μm, such as 12.4 μm, 13.6 μm, 14.2 μm, or 16.3 μm; a wetting agent, such as sorbitan monolaurate; an emulsifier, such as polyoxyl-35 castor oil or polyethylene glycol (15)-hydroxystearate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In yet further embodiments, the formulation comprises, consists essentially of, or consists of a combination of LNGB having a median particle size ranging from 11 μm to 15 μm, such as 12.4 μm, 13.6 μm, or 14.2 μm; a wetting agent, such as sorbitan monolaurate; an emulsifier, such as polyoxyl-35 castor oil or polyethylene glycol (15)-hydroxystearate; a thickening agent, such as sodium carboxymethyl cellulose; two buffer compounds, such as sodium phosphate dibasic and sodium dihydrogen phosphate dihydrate; a preservative, such as benzyl alcohol; and sterile WFI USP.

In any of the embodiments wherein the formulation consists essentially of the above-listed components, the formulation is free of components that are considered in the art as being toxic to subjects and/or that decrease stability of the particles (such as by causing aggregation or degradation). Representative formulation embodiments also are described in the Examples section, herein.

### Method Embodiments

Also disclosed herein are embodiments of a method of making formulation embodiments described herein. Method embodiments of the present disclosure are conveniently carried out on large scale and can be conducted in a single manufacturing location, whereas other methods in the art require conducting separate method steps in different/separate locations of different geographic locations due to the need for specialized machinery and/or operating parameters. Method embodiments disclosed herein also provide the ability to control particle size of the LNGB particles such that stable and/or more concentrated particle formulations can be made.

In particular embodiments, the method comprises producing the LNGB particles prior to combining the LNGB particles with the one or more pharmacologically acceptable excipients. In particular embodiments, the LNGB particles are prepared by sterile filtration followed by spray drying to produce sterile particles having controlled median particle size. Sterile filtration provides a filtrate comprising the levonorgestrel butanoate and particles of the levonorgestrel butanoate can be produced from the filtrate by spray drying the filtrate. In an independent embodiment, spray drying can be replaced with micronization (e.g., dry or wet milling or grinding). Such independent method embodiments may further comprise an intermediate drying or crystallization step prior to micronization. Method embodiments described herein, however, do not comprise using a sterilization step, such as an irradiation-based sterilization technique (e.g., gamma or e-beam irradiation), as such techniques result in producing impurities that render formulations unsuitable for administration to subjects. Method embodiments comprising spray drying as described herein also do not comprise a recrystallization step.

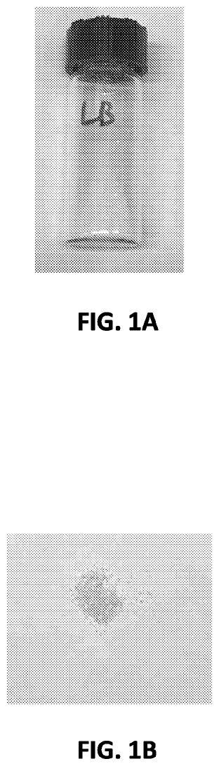

In particular embodiments, the method consists of, or consists essentially of, sterile filtration and spray drying. In particular embodiments, the method consists of, or consists essentially of, sterile filtration, drying and/or recrystallization, and micronization. If the method consists essentially of sterile filtration and spray drying, the method does not include using gamma or e-beam irradiation sterilization, as such sterilization techniques result in producing impurities that render the formulation unsuitable for administration to subjects. For example, see FIGS. 1A and 1B, which shows that terminal irradiation sterilization of LNGB particles, such as gamma or e-beam irradiation, produces impurities that cause LNGB particles (FIG. 1A) to have a yellow color (FIG. 1B). These impurities also are identified using high-performance liquid chromatography (HPLC). Exemplary such HPLC traces are shown in FIGS. 3-5, which show that the impurities cannot be removed or prevented even by purging with inert gases. This color and impurity presence renders the LNGB particles unsuitable for administration under FDA requirements and specifications.

Sterile filtration can comprise filtering the LNGB using a solvent, such as an ether-based solvent. In some embodiments, the solvent is tetrahydrofuran (“THF”), 2-methyl THF, and the like. The LNGB is filtered through an autoclaved membrane filter using the solvent and isolated as a filtrate. In some embodiments, the membrane filter is a 0.2 μm PTFE membrane filter. If tubing is used during the filtration process, it typically is a chemically-resistant tubing that exhibits chemical resistance against the solvent used during filtration.

In particular embodiments, spray drying comprises utilizing a flow rate, inlet temperature, atomization spray pressure, and blower speed to produce the LNGB particles from the filtrate obtained from the sterile filtration. In particular embodiments, the flow rate ranges from 4 mL/minute to 6 mL/minute, such as 4 mL/minute, 5 mL/minute, 6 mL/minute, or fractions thereof. The spray drying inlet temperature ranges from 120° C. to 145° C., such as 130° C. to 140° C., or 130° C. to 132° C. The atomization spray pressure ranges from 0.05 mPa to 0.1 mPa, such as 0.05 mPa to 0.08 mPa, or 0.06 mPa, 0.07 mPa, or 0.08 mPa. The blower speed ranges from 0.3 m³/minute to 0.6 m³/minute, such as 0.4 m³/minute to 0.55 m³/minute, or 0.45 m³/minute to 0.5 m³/minute, or 0.49 m³/minute. In particular embodiments, the flow rate is 5 mL/min, the spray drying inlet temperature is 132° C., the atomization spray pressure is 0.07 mPa, and the blower speed 0.49 m³/minute.

The method further comprises combining the LNGB particles with one or more pharmaceutically acceptable excipients described herein. In some embodiments, the LNGB particles are combined with the pharmaceutically acceptable excipient by producing a first solution comprising one or more of the pharmaceutically acceptable excipient(s) and a second solution comprising the LNGB particles. In some embodiments, the first solution can comprise a thickening agent, a preservative, a buffer component, or any combination thereof; and sterile water. In some embodiments, the second solution can comprise the LNGB particles; sterile water; and any wetting agent(s), dispersing agent(s), and/or emulsifier(s). The first solution and the second solution are then combined in any order (such as by adding the first solution to the second solution, or adding the second solution to the first solution) to provide the formulation. In yet other embodiments, a single solution of the LNGB particles, sterile water, the wetting agent(s), the dispersing agent(s), the thickening agent, the buffer component, the preservative, and any optional emulsifier. In some embodiments, the formulation and/or the first and/or second solutions can be homogenized to provide a uniform dispersion.

Also disclosed herein are methods of using the formulation embodiments. In particular embodiments, the method comprises using the formulation as a contraceptive formulation to provide long lasting, reversible contraception effect for subjects, particularly female subjects. The disclosed formulation embodiments are stable and exhibit suitable contraceptive effects, such as ovulation suppression, for at least two months, and more typically more than two months, such as at least three months, or four months, five months, and even six months. Further, disclosed formulation embodiments exhibit desirable activity without any significant negative impact on liver function during treatment and/or recovery. And, side effects typically seen in conventional contraceptives, such as Depo-Provera®, can be avoided or at least significantly minimized with the disclosed formulation embodiments, particularly since less hormone is needed for contraceptive effects. Such side effects can include weight gain and mood swings. In additional embodiments, the method can comprise using the formulation to treat or prevent progestin-related diseases or conditions, such as dysfunctional uterine bleeding, endometrial hyperplasia, endometriosis, fibroids, pre-menstrual syndrome, cancer, and other reproductive system/tissue/tract disorders or conditions.

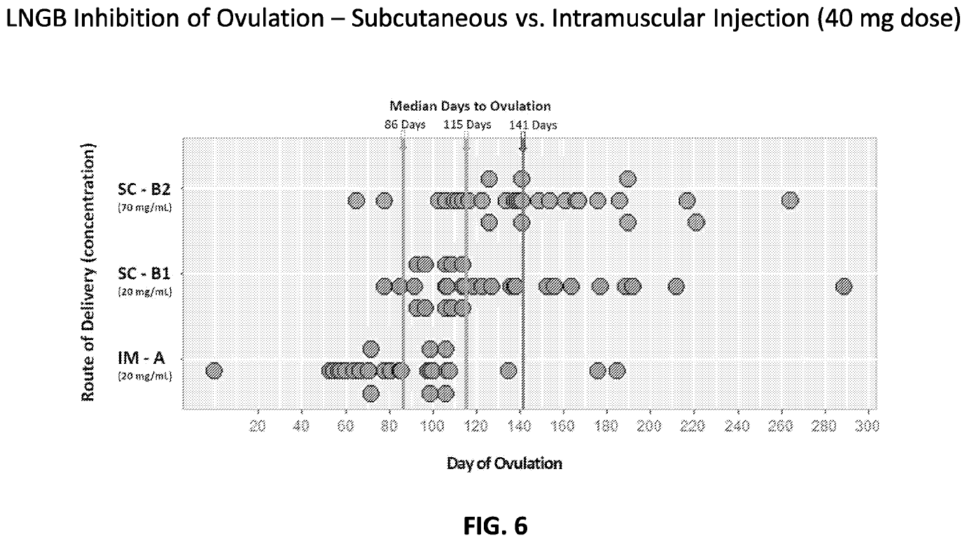

The formulation embodiments are suitable for different types of administration techniques including, but not limited to, injection methods, such as subcutaneous injection (or “SC” injection) or intramuscular injection (or “IM” injection). The formulation embodiments also have the benefit of being suitable for self-administration. In particular embodiments, the formulation exhibits superior contraceptive activity if administered via subcutaneous injection. For example, FIG. 6 provides a graph comparing results obtained from intramuscular injection versus subcutaneous injection. As can be seen in FIG. 6, the particular formulation embodiment evaluated in this example exhibited a longer lasting contraceptive effect (or “return to ovulation” time period) when administered subcutaneously as opposed to intramuscularly. The formulation embodiments of FIG. 6 comprised LNGB having a median particle size ranging from 10 μm to 16 μm; a wetting agent and a dispersing agent, namely polysorbate 80 and sorbitan monopalmitate; a thickening agent, namely sodium carboxymethyl cellulose; two buffer compounds, namely sodium phosphate dibasic and sodium dihydrogen phosphate dihydrate; a preservative, namely benzyl alcohol; and sterile WFI USP.

In particular embodiments, the method of use is a method of providing contraception to a subject, comprising administering a dosage of levonorgestrel butanoate by administering a formulation embodiment of the present disclosure to the subject via injection. In some embodiments, injection comprises subcutaneous injection. In other embodiments, injection comprises intramuscular injection. In yet other particular embodiments, the method of use is a method of treating or preventing a progestin-related disease or condition in a subject, comprising administering a formulation embodiment of the present disclosure to the subject.

### Overview of Several Embodiments

Disclosed herein are embodiments of a pharmaceutical formulation, comprising: levonorgestrel butanoate particles having a median particle size ranging from 10 μm to 16 μm; a wetting agent; and a dispersing agent, an emulsifier, or a combination thereof; wherein the levonorgestrel butanoate is present at a concentration ranging from greater than 20 mg/mL to a concentration of 100 mg/mL and provided that if the pharmaceutical formulation comprises a wetting agent and a dispersing agent, then the wetting agent and the dispersing agent are present at a ratio of 2:0.1 to 6:0.1 (wetting agent: dispersing agent).

In any or all of the above embodiments, the levonorgestrel butanoate particles have a median particle size ranging from greater than 12.6 μm to a median particle size of 15 μm.

In any or all of the above embodiments, the levonorgestrel butanoate particles have a median particle size of 13 μm to 14 μm.

In any or all of the above embodiments, the formulation comprising the wetting agent and the dispersing agent and the ratio of wetting agent to dispersing agent is 3:0.1 to 5:0.1.

In any or all of the above embodiments, the formulation is formulated to administer a dose of levonorgestrel butanoate that ranges from 30 mg to 90 mg.

In any or all of the above embodiments, the formulation provides a contraceptive effect for at least two months to six months.

In any or all of the above embodiments, the formulation provides a contraceptive effect for at least two months, at least three months, at least four months, at least five months, or at least six months.

In any or all of the above embodiments, the wetting agent is a polysorbate surfactant selected from polysorbate 20, polysorbate 40, polysorbate 60, or polysorbate 80.

In any or all of the above embodiments, the dispersing agent is a sorbitan surfactant selected from sorbitan monolaurate, sorbitan monopalmitate, sorbitan monostearate, sorbitan monooleate, sorbitan tristearate, sorbitan sesquioleate, sorbitan trioleate; a sulfate surfactant; or any combination thereof.

In any or all of the above embodiments, the formulation further comprises a thickening agent selected from carboxymethyl cellulose and/or salt forms thereof; methyl cellulose, hydroxypropylcellulose, hydroxyethylcellulose, hydroxypropylmethylcellulose, and/or crosslinked forms thereof; polyvinyl alcohol; polyvinylpyrrolidone; acacia; gelatin; or any combination thereof.

In any or all of the above embodiments, the formulation further comprises a buffer component selected from a phosphate buffer, an acetate buffer, a citrate buffer, a tartrate buffer, an amine-based buffer, or any combination thereof.

In any or all of the above embodiments, the formulation further comprises a preservative selected from a paraben compound, a phenylmercuric compound, a benzyl compound, an aromatic compound, an alkyl halide, or any combination thereof.

In any or all of the above embodiments, the levonorgestrel butanoate particles having a median particle size of 13 μm to 14.5 μm; the wetting agent is polysorbate 80; and the dispersing agent is present and is sorbitan monopalmitate.

In any or all of the above embodiments, the levonorgestrel butanoate particles having a median particle size of 13.5 μm to 14 μm; the wetting agent is polysorbate 80; and the dispersing agent is present and is sorbitan monopalmitate.

In any or all of the above embodiments, the levonorgestrel butanoate particles having a median particle size of 13.7 μm; the wetting agent is polysorbate 80; and the dispersing agent is present and is sorbitan monopalmitate.

In any or all of the above embodiments, the formulation further comprises sodium carboxymethyl cellulose, sodium phosphate dibasic, sodium dihydrogen phosphate dihydrate, and benzyl alcohol.

In any or all of the above embodiments, the emulsifier is present and is an ionic or non-ionic surfactant.

In any or all of the above embodiments, the pharmaceutical formulation comprises levonorgestrel butanoate particles having a median particle size ranging from 12 μm to 15 μm; a wetting agent; a dispersing agent, an emulsifier, or a combination thereof; a thickening agent; a buffer component; and a preservative, wherein the levonorgestrel butanoate is present at a concentration ranging from greater than 20 mg/mL to a concentration of 100 mg/mL and provided that if the pharmaceutical formulation comprises a wetting agent and a dispersing agent, then the wetting agent and the dispersing agent are present at a ratio of 3:0.1 to 5:0.1 (wetting agent: dispersing agent).

In any or all of the above embodiments, pharmaceutical formulation comprises the levonorgestrel butanoate particles having a median particle size of 13 μm to 14.5 μm; the wetting agent is polysorbate 80; the dispersing agent is present and is sorbitan monopalmitate; the thickening agent is sodium carboxymethyl cellulose; the buffer component is a combination of sodium phosphate dibasic and sodium dihydrogen phosphate dihydrate; and the preservative is benzyl alcohol.

In any or all of the above embodiments, pharmaceutical formulation comprises the levonorgestrel butanoate particles having a median particle size of 13.5 μm to 14 μm; the wetting agent is polysorbate 80; the dispersing agent is present and is sorbitan monopalmitate; the thickening agent is sodium carboxymethyl cellulose; the buffer component is a combination of sodium phosphate dibasic and sodium dihydrogen phosphate dihydrate; and the preservative is benzyl alcohol.

In any or all of the above embodiments, the formulation comprises levonorgestrel butanoate particles having a median particle size ranging from 11 μm to 15 μm; at least one dispersing agent; a thickening agent; a buffer component; and a preservative, wherein the levonorgestrel butanoate is present at a concentration ranging from greater than 20 mg/mL to a concentration of 100 mg/mL.

In any or all of the above embodiments, the levonorgestrel butanoate particles have a median particle size ranging from greater than 11.3 μm to a median particle size of 14.2 μm.

In any or all of the above embodiments, the dispersing agent is sorbitan monopalmitate.

In any or all of the above embodiments, the formulation is formulated to administer a dose of levonorgestrel butanoate that ranges from 30 mg to 90 mg.

Also disclosed are embodiments of a method of making a pharmaceutical formulation according to any or all of the above embodiments, the method comprising: subjecting levonorgestrel butanoate to sterile filtration to provide a filtrate comprising sterile levonorgestrel butanoate; producing levonorgestrel butanoate particles from the filtrate; and combining the levonorgestrel butanoate particles with one or more of the wetting agent, the dispersing agent, the emulsifier, or the combination thereof; wherein the method does not comprise using irradiation for any sterilization.

In any or all of the above embodiments, producing levonorgestrel butanoate particles from the filtrate comprises spray drying the filtrate.

In any or all of the above embodiments, spray drying the filtrate controls the median particle size of the levonorgestrel butanoate particles.

Also disclosed are embodiments of a method of making a pharmaceutical formulation according to any or all of the above embodiments, the method comprising: micronizing levonorgestrel butanoate to provide the levonorgestrel butanoate particles and combining the levonorgestrel butanoate particles with one or more of the wetting agent, the dispersing agent, the emulsifier, or the combination thereof; wherein the method does not comprise using irradiation for any sterilization.

In any or all of the above embodiments, the method does not comprise using gamma or e-beam irradiation.

Also disclosed are embodiments of a method of providing contraception to a subject, comprising administering a dosage of levonorgestrel butanoate ranging from 20 mg to 140 mg by administering the formulation of any or all of the above formulation embodiments to the subject via injection.

In any or all of the above embodiments, injection comprises subcutaneous injection.

In any or all of the above embodiments, injection comprises intramuscular injection.

In any or all of the above embodiments, the concentration of levonorgestrel butanoate in the formulation is 70 mg/mL.

Also disclosed are embodiments of a method of treating or preventing a progestin-related disease or condition in a subject, comprising administering the formulation of any or all of the above formulation embodiments to the subject.

### EXAMPLES

### Example 1

In this example, spray dried, micronized LNGB particles were prepared. The LNGB was prepared in an ISO 5 clean room using a Yamato Versatile Spray Dryer GB-210A fitted with a Yamato Organic Solvent Recovery Unit GAS410, Watson Marlow peristaltic pump 520DiN, two-way nozzle system (0.4 mm ID), and custom designed flexible film isolator. Prior to spray drying, 5% LNGB in tetrahydrofuran (THF) was sterile filtered through an autoclaved 0.2 μm PTFE membrane filter. Gore® Sta-Pure® laminated PTFE tubing was used for chemical resistance to THF. The following controlled settings were used: flow rate ˜5 mL/min, spray drying inlet temperature 132° C., atomization spray pressure 0.07 mPa, and blower speed 0.49 m³/minute. Release criteria included analysis for elemental impurities (using USP<232, 233>, the relevant portion of which is incorporated herein by reference) residual solvents per validated GC method, X-ray powder diffraction (XRPD) (using USP<941>, the relevant portion of which is incorporated herein by reference), particle size distribution (using USP<429>, the relevant portion of which is incorporated herein by reference), and sterility (using USP<71>, the relevant portion of which is incorporated herein by reference).

Two formulation embodiments were made using the spray-dried particles of LNGB obtained from the above process. The contents and amounts are provided in Table 1, below.

TABLE 1

| | |
|---|---|
| #1 | #2 |

| | | | | |
|---|---|---|---|---|
| Levonorgestrel Butanoate | 20 | mg/mL | 70 | mg/mL |
| Concentration | | | | |
| Particle Size - Dv (50) | 12.6 | μm | 13.7 | μm |

Components

| | #1 | #2 |
|---|---|---|
| Ingredient | % (w/v) | % (w/v) |
| Levonorgestrel Butanoate | 2.0 | 7.0 |
| Sodium Phosphate Dibasic | 0.3 | 0.3 |
| Sodium Dihydrogen Phosphate | 1.49 | 1.49 |
| Dihydrate | | |
| Sodium Carboxymethylcellulose | 0.5 | 0.8 |
| Benzyl Alcohol, NF | 1.03 | 1.03 |
| Super-refined Polysorbate 80-LQ-(MH) | 0.2 | 10 |
| Span 40-PW-(AP) | 0.2 | 0.2 |
| Sterile Water for Injection, USP | Q.S. to 100% | Q.S. to 100% |
| Total | 100 | 100 |

### Example 2

In this example, an exemplary phase I study is described for evaluating efficacy of formulation embodiments disclosed herein. While this example describes two particular formulation embodiments, other formulation embodiments disclosed herein are equally applicable.

Women subjects, having a normal body weight (e.g., BMI<30 kg/m²) or that are obese (e.g., a BMI≥30 kg/m² and <40 kg/m²), are divided into 5 groups using two formulation embodiments having different concentrations of the LNGB. Three groups receive LNGB as a subcutaneous (SC) injection and the other two groups receive LNGB as an intramuscular (IM) injection. The initial dose administered is 40 mg using 2 mL of a 20 mg/mL concentration. A repeat (or second stage dose) of the 40 mg SC dose is done with either 2 mL of a 20 mg/mL concentration or 0.57 mL of a 70 mg/mL concentration. In yet other examples, a different dose of 50 mg is administered using 0.71 mL of a 70 mg/mL concentration. In yet other examples, a different dose of 60 mg is administered using 0.86 mL of a 70 mg/mL concentration. The SC injections are performed slowly under the skin in the abdomen to ensure drug is dispensed into the tissue layer between the skin and the muscle following standard procedures for subcutaneous injections. For IM injections, the formulation is slowly injected in the deltoid avoiding the injection into blood vessels following standard procedures for intramuscular injections; however, the SC injection may be given in a different location than the abdomen and the IM injection in a different location than the deltoid.

The first formulation to be used in this example is an LNGB injectable suspension at a concentration of 40 mg/2 ml (20 mg/mL). This formulation comprises a suspension of spray dried LNGB particles, with an approximate median particle size of 13 microns, in an aqueous vehicle containing the following pharmaceutically acceptable excipients: super refined polysorbate 80 (Tween® 80), spray dried sorbitan monopalmitate (Span® 40), sodium carboxymethylcellulose, sodium phosphate dibasic anhydrous, sodium phosphate monobasic dihydrate, benzyl alcohol and WFI, USP. Each vial contains 2 mL at 20 mg/mL for a total of 40 mg.

The second formulation to be used in this example is an LNGB injectable suspension at a concentration of 140 mg/2 mL (70 mg/mL). This higher concentration formulation comprises a suspension of spray dried LNGB particles, with an approximate median particle size of 13.7 micron, in an aqueous vehicle containing the following pharmaceutically acceptable excipients: super refined polysorbate 80 (Tween® 80), spray dried sorbitan monopalmitate (Span® 40), sodium carboxymethylcellulose, sodium phosphate dibasic anhydrous, sodium phosphate monobasic dihydrate, benzyl alcohol and WFI, USP. Each vial contains 2 mL at 70 mg/mL for a total of 140 mg. Doses of 40 mg can be administered using 0.57 mL of the 2 mL total. Doses of 50 mg can be administered using 0.71 mL of the 2 mL total. Doses of 60 mg can be administered using 0.86 mL of the 2 mL total.

Subjects are evaluated using the following pharmacokinetic/pharmacodynamic parameters:
LNG PK levels;
Serum progesterone (P4), luteinizing hormone (LH), estradiol (E2), follicle stimulating hormone (FSH), sex hormone binding globulin (SHBG);
Ovarian follicular activity assessed by transvaginal ultrasound;
Endometrial thickness assessed by transvaginal ultrasound; and
Bleeding Profile, wherein the bleeding patterns (number of days of bleeding, spotting, no bleeding, length of longest episode, and intensity of bleeding) are analyzed over 30-day and 90-day reference periods. After the return to menses, the characteristics of the menstrual cycle is recorded (length, intensity, duration of bleeding, symptoms).

The relationship between LNG levels following injection of LNGB by different routes of delivery (IM and SC) is evaluated, along with the impact on ovulation as the level declines over several weeks. Other endpoints that can be evaluated include return to ovulation and normal menstrual cycles. The assessment of these changes in ovarian function and hormone profiles are then correlated with LNG plasma concentration levels achieved after administration of the injection. The impact of BMI on study outcomes also is evaluated. In particular embodiments, blood samples to evaluate platelets, differential (RBC, WBC, Hemoglobin, Hematocrit, % Basophils, % Eosinophils, % Lymphocytes, % Monocytes, % Neutrophils, and Platelets), and clinical chemistries are determined, including a basic metabolic panel and lipid profiles (glucose, creatinine, calcium, potassium, sodium, chloride, BUN, total cholesterol, HDL, LDL, and triglycerides). Additionally, liver function tests (SGPT, SGOT, albumin, total protein, total bilirubin, direct bilirubin, and GGT) are completed on collected blood samples. Hormone levels also are evaluated, including progesterone, luteinizing hormone (LH), estradiol (E2), follicle stimulating hormone (FSH), sex hormone binding globulin (SHBG), and LNG PK levels. Also, transvaginal ultrasound examinations are used to measure endometrial stripe thickness, appearance of endometrium, measurement of all follicles≥10 mm on each ovary and assessment of the dominant follicle.

Results from evaluations of the above-mentioned metrics are evaluated on a grading scale of systemic quantitative adverse events, subjected adverse events, and/or laboratory test abnormalities, wherein the following scale is used:
Mild—events require minimal or no treatment and do not interfere with the subject's daily activities.
Moderate—events result in a low level of inconvenience or concern with the therapeutic measures. Moderate events may cause some interference with functioning.
Severe—events interrupt a subject's usual daily activity and may require systemic drug therapy or other treatment. Severe events are usually incapacitating.

Exemplary grading rubrics are provided in Tables 2-4.

TABLE 2

| Systemic | Mild | Moderate | Severe |
|---|---|---|---|
| (Quantitative) | (Grade 1) | (Grade 2) | (Grade 3) |
| Fever (° C.)* | ≥37.8-<38 | ≥38-<39 | ≥39 |
| Tachycardia, beats per | 101-115 | 116-130 | ≥131 |
| minute † | | | |
| Bradycardia, beats per | 49-45 | 44-40 | <40 |
| minute [when awake] | | | |
| Hypertension (systolic), mmHg | 141-160 | 161-180 | ≥180 |
| Hypertension (diastolic), mmHg | 91-100 | 101-110 | ≥110 |
| Hypotension (systolic), mmHg | 89-85 | 84-80 | ≤79 |
| PR Interval (prolonged) | PR interval | PR interval > | Type II 2^{nd} degree |
| | 0.21-.025 sec | 0.25 | AV block OR Ventricular |
| | | | pause > 3.0 sec |

TABLE 3

| Systemic | Mild | Moderate | Severe |
|---|---|---|---|
| (Subjective) | (Grade 1) | (Grade 2) | (Grade 3) |
| Headache | No interference | Some interference | Significant interference, |
| | with activity | with activity | prevents daily activity |
| | | | for ≥1 day |
| Fatigue/Malaise | No interference | Some interference | Significant interference, |
| | with activity | with activity | prevents daily activity |
| | | | for ≥1 day |
| Myalgia/Body | No interference | Some interference | Significant interference, |
| Ache | with activity | with activity | prevents daily activity |
| | | | for ≥1 day |
| Nausea | No interference | Some interference | Significant interference, |
| | with activity | with activity | prevents daily activity |
| | | | for ≥1 day |

TABLE 4

| | Mild | | Moderate | Severe | |
|---|---|---|---|---|---|
| Laboratory | (Grade 1) | | (Grade 2) | (Grade 3) | |
| Hgb -change from baseline | <1.0 | gm/dL | >1.0 and <3.0 | >3.0 | gm/dL |
| value in gm/dL | | | gm/dL | | |
| WBC cells/mm³ (Increase in WBC to | <2000 | cells/mm³ | >2000 to <4000 | >4000 | cells/mm³ |
| greater than) from baseline | | | cells/mm³ | | |
| WBC cells/mm³ (Decrease in WBC to | <1000 | cells/mm³ | >1000 to <3000 | >3 cells/mm³000 | |
| less than) from baseline | | | cells/mm³ | | |
| Platelets -cell/mm³ | <75000 | cells/mm³ | >75000 to <150,000 | >150,000 | cells/mm³ |
| from baseline | | | cells/mm³ | | |

| | | | |
|---|---|---|---|
| SGPT (increase by factor) | >2 & <3 × ULN | ≥3 & <4 × ULN | ≥4 × ULN |
| SGOT (increase by factor) | >2 & <3 × ULN | ≥3 & <4 × ULN | ≥4 × ULN |
| GGT (increase by factor) | >2 & <3 × ULN | ≥3 & <4 × ULN | ≥4 × ULN |
| Serum creatinine | <1.0 mg/dL | 1.0 to 3.0 mg/dL | >3.0 mg/dL |
| | above normal | above normal | above normal |
| Urine Protein | 1+ | 2+ | 3, 4+ |
| Urine Glucose | 1+ | 2+ | >3+ |
| Urine RBC | Few | >10 per hpf | Many per hpf |

### Example 3

This example provides additional exemplary formulation embodiments. The contents of the formulation embodiments of this example are described in Tables 5 and 6. Evaluation techniques described in Example 2 can be used for these additional formulations. In particular examples, a white uniform suspension is obtained when the LNGB particles are included in the formulation and the formulation remains homogenous for a desirable period of time.

TABLE 5

| | LNGB Concentration (mg/mL) | | | | | |
|---|---|---|---|---|---|---|
| | 20 | 50 | 50 | 50 | 50 | 50 |
| | Formula | Formula | Formula | Formula | Formula | Formula |
| | #1 | #2 | #3 | #4 | #5 | #6 |
| Ingredient | % w/v | % w/v | % w/v | % w/v | % w/v | % w/v |
| Sodium Phosphate Dibasic | 0.3 | 0.3 | 0.3 | 0.3 | 0.3 | 0.3 |
| Sodium Dihydrogen Phosphate | 1.49 | 1.49 | 1.49 | 1.49 | 1.49 | 1.49 |
| Dihydrate | | | | | | |
| Sodium Carboxymethylcellulose | 1 | 1 | 0.6 | 0.5 | 0.5 | 0.5 |
| Benzyl Alcohol, NF | 1.03 | 1.03 | 1.03 | 1.03 | 1.03 | 1.03 |
| Tween ® 80 | — | — | — | — | 7 | — |
| Span ® 40 | 0.2 | 0.2 | 0.2 | 0.2 | 0.1 | 0.1 |
| Sodium lauryl sulfate | 0.7 | 0.7 | — | — | — | — |
| Kolliphor ® EL | — | — | 7 | — | — | 5 |
| Kolliphor ® HS | — | — | — | 4.4 | — | — |
| Span ® 20 | — | — | — | — | 0.1 | 0.1 |
| Castor oil | | | | | | |
| Sterile WFI USP | Q.S. to | Q.S. to | Q.S. to | Q.S. to | Q.S. to | Q.S. to |
| | 100% | 100% | 100% | 100% | 100% | 100% |
| Total | 100 | 100 | 100 | 100 | 100 | 100 |
| Spray Dried Powder Particle Size - D₅₀ | 13.6 | 16.3 | 14.2 | | | |
| Suspension Particle Size - D₅₀ | 11.6 | 14.2 | 11.3 | | | |

TABLE 6

| | LNGB Concentration (mg/mL) | | | | |
|---|---|---|---|---|---|
| | 50 | 50 | 50 | 50 | 50 |
| | Formula | Formula | Formula | Formula | Formula |
| | #7 | #8 | #9 | #10 | #11 |
| Ingredient | % w/v | % w/v | % w/v | % w/v | % w/v |
| Sodium Phosphate Dibasic | 0.3 | 0.3 | 0.3 | 0.3 | 0.3 |
| Sodium Dihydrogen Phosphate | 1.49 | 1.49 | 1.49 | 1.49 | 1.49 |
| Dihydrate | | | | | |
| Sodium Carboxymethylcellulose | 0.5 | 0.5 | 0.6 | 0.5 | 0.7 |
| Benzyl Alcohol, NF | 1.03 | 1.03 | 1.03 | 1.03 | |
| Tween ® 80 | — | 6 | — | — | 10 |
| Span ® 40 | 0.1 | — | — | — | 0.2 |
| Sodium lauryl sulfate | — | — | — | — | |
| Kolliphor ® EL | — | — | 3 | — | |
| Kolliphor ® HS | 3 to 4 | — | — | 3 | |
| Span ® 20 | 0.1 | 0.2 | 0.2 | 0.2 | |
| Castor oil | | | | | |
| Sterile WFI USP | Q.S. | Q.S. | Q.S. | Q.S. | Q.S. |
| | to 100% | to 100% | to 100% | to 100% | to 100% |
| Total | 100 | 100 | 100 | 100 | 100 |
| Spray Dried Powder | | | 14.3 | | 15.4 |
| Particle Size - D₅₀ | | | | | |
| Suspension Particle | | | 12.4 | | 13.2 |
| Size - D₅₀ | | | | | |

### Example 4

In this example, median days to ovulation were evaluated using formulation embodiments having differences in concentration and/or particle size as well as comparing different administration routes using these formulations. In particular, Formulations #1 and #2 from Example 1 were evaluated in subjects and comparisons made between results obtained from the different formulations using two administration routes, subcutaneous and intramuscular. Results are illustrated in FIG. 6, which is a plot showing the number of days to ovulation as a function of administration route and concentration, wherein the routes of administration included an intramuscular stage A (referred to as “IM-A”), a subcutaneous stage B1 (referred to as “SC-B1”), and a subcutaneous stage B2 (referred to as “SC-B2”). Observations obtained from the results illustrated in FIG. 6 are provided below.

As can be seen in FIG. 6, the median days to ovulation were significantly different using the different routes of administration for the formulation having a 20 mg/mL concentration and using a 40 mg dose (comparing data points for SC-B1 and IM-A). In particular, the median days to ovulation using SC-B1 amounted to 115 days, as compared with 86 days for IM-A. These results are significantly different and represent a difference of 29 days to ovulation for embodiments having an identical injection volume, dose, concentration, and formulation; the only difference being the route of administration. The longer 29-day period to reach median ovulation using SC injection was highly unexpected.

FIG. 6 further illustrates that a significant difference in median days to ovulation using a different route of administration for the two formulations having an identical dose of 40 mg, but with a difference in concentration (i.e., 70 mg/mL, administered via the SC route and 20 mg/mL administered via the IM route). By comparing data points for SC-B2 and IM-A, it can be seen that the median days to ovulation were significantly different using the different route of administration. In particular, the median days to ovulation using SC-B2 amounted to 141 days, as compared with 86 days for IM-A. These results are significantly different and represent a difference of 60 days to ovulation for embodiments having an identical dose but having different routes of administration (subcutaneous vs. intramuscular) and injection concentration (70 mg/mL vs. 20 mg/mL). In view of the results comparing SC-B1 and IM-A, it was anticipated that the same longer 29-day period to reach median ovulation would be observed in SC-B2 (as compared with IM-A); however, the result of a longer 60-day period to reach median ovulation using the SC injection route was highly unexpected.

As can also be seen from FIG. 6, a significant difference in median days to ovulation was observed when comparing the results for the formulations having an identical dose of 40 mg and using the same route of administration, but with a difference in concentration (comparing data points for SC-B2 and SC-B1). The median days to ovulation using SC-B2 amounted to 141 days, as compared with 115 days for SC-B1. These results are significantly different and represent a difference of 26 days to ovulation for embodiments having an identical dose but different concentration. The reduction in injection volume using 70 mg/ml formulation shows significant delay in days to ovulation, which was highly unexpected. These results also establish that particle size can have an effect on median days to ovulation as the SC-B2 formulation comprises LNGB particles with a larger average particle size as compared with SC-B1.

### Comparative Example 1

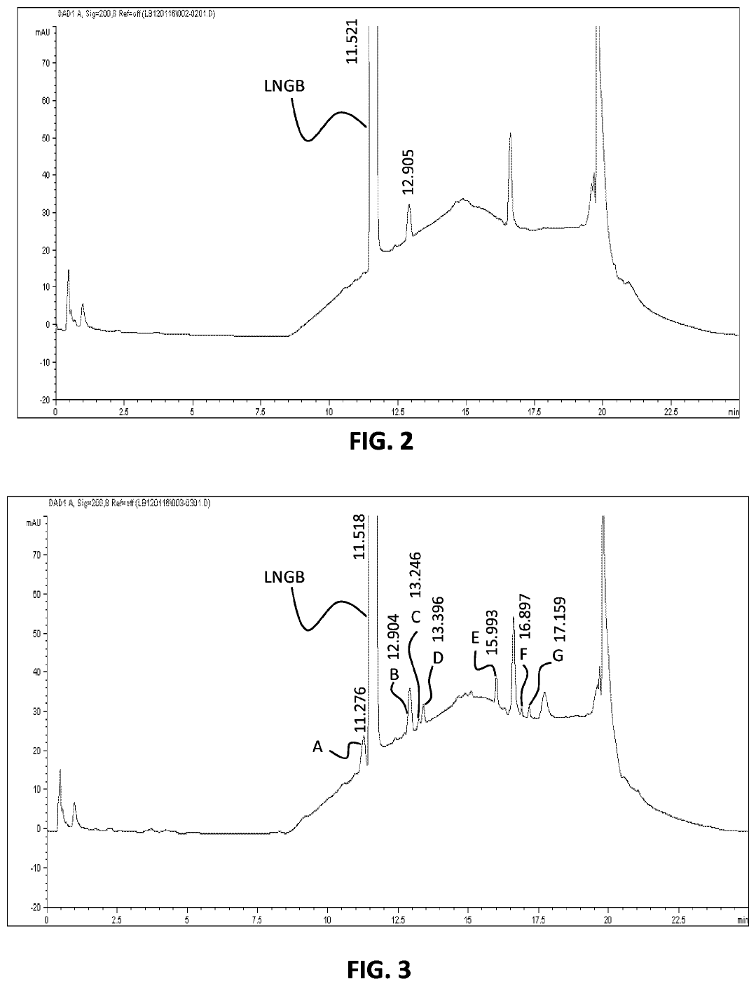

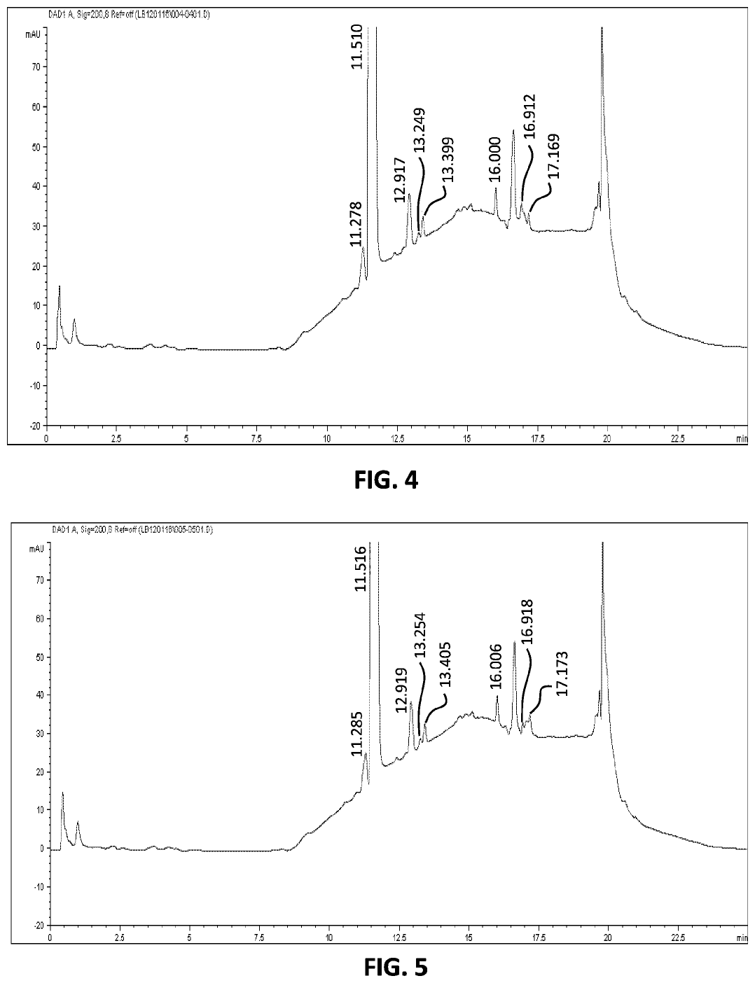

In this comparative example, a sample of LNGB particles was prepared using a gamma irradiation step to sterilize spray dried particles. The LNGB particles were obtained by spray drying and then were subjected to gamma irradiation to evaluate the effect of irradiation on the particles. The resulting particles exhibited a yellow color, due to impurities present in the sample after gamma irradiation. FIG. 1B shows the yellow colored sample as compared to the initial white/translucent particles prior to gamma irradiation (FIG. 1A). Qualitative HPLC was performed to compare the peak profiles of the LNGB particles before and after irradiation. FIGS. 2-5 show HPLC traces of the sample before (FIG. 2) and after (FIGS. 3-5) irradiation. Irradiated samples generated three major peaks, A, D, and E, at >0.15%, as well as a small peak, C, at ˜0.07%. Peaks F and G were observed in an un-purged irradiated sample at low amounts (FIG. 3). In N₂- and Ar-purged irradiated samples (FIGS. 4 and 5, respectively), peaks F and G were not resolved from other peaks (either degradation products or gradient artifacts).

In view of the many possible embodiments to which the principles of the present disclosure may be applied, it should be recognized that the illustrated embodiments are only preferred examples and should not be taken as limiting the scope of the disclosure. Rather, the scope is defined by the following claims. We therefore claim as our invention all that comes within the scope and spirit of these claims.

1. A pharmaceutical formulation, comprising:
levonorgestrel butanoate particles having a median particle size ranging from 10 μm to 16 μm;
a wetting agent; and
a dispersing agent, an emulsifier, or a combination thereof;
wherein the levonorgestrel butanoate is present at a concentration ranging from greater than 20 mg/ml to a concentration of 100 mg/mL and provided that if the pharmaceutical formulation comprises a wetting agent and a dispersing agent, then the wetting agent and the dispersing agent are present at a ratio of 2:0.1 to 6:0.1 (wetting agent: dispersing agent).

2. (canceled)

3. The pharmaceutical formulation of claim 1, wherein the levonorgestrel butanoate particles have a median particle size of 13 μm to 14 μm.

4. The pharmaceutical formulation of claim 1, wherein the formulation comprises the wetting agent and the dispersing agent and the ratio of wetting agent to dispersing agent is 3:0.1 to 5:0.1.

5. The pharmaceutical formulation of claim 1, wherein the formulation is formulated to administer a dose of levonorgestrel butanoate that ranges from 30 mg to 90 mg and the formulation provides a contraceptive effect for at least two months to six months.

6-7. (canceled)

8. The pharmaceutical formulation of claim 1, wherein the wetting agent is a polysorbate surfactant selected from polysorbate 20, polysorbate 40, polysorbate 60, or polysorbate 80.

9. The pharmaceutical formulation of claim 1, wherein the dispersing agent is a sorbitan surfactant selected from sorbitan monolaurate, sorbitan monopalmitate, sorbitan monostearate, sorbitan monooleate, sorbitan tristearate, sorbitan sesquioleate, sorbitan trioleate; a sulfate surfactant; or any combination thereof.

10. The pharmaceutical formulation of claim 1, further comprising (i) a thickening agent selected from carboxymethyl cellulose and/or salt forms thereof; methyl cellulose, hydroxypropylcellulose, hydroxyethylcellulose, hydroxypropylmethylcellulose, and/or crosslinked forms thereof; polyvinyl alcohol; polyvinylpyrrolidone; acacia; gelatin; or any combination thereof; (ii) a buffer component selected from a phosphate buffer, an acetate buffer, a citrate buffer, a tartrate buffer, an amine-based buffer, or any combination thereof; (iii) a a preservative selected from a paraben compound, a phenylmercuric compound, a benzyl compound, an aromatic compound, an alkyl halide, or any combination thereof; or (iv) any combination of two or more of (i)-(iii).

11-12. (canceled)

13. The pharmaceutical formulation of claim 1, wherein:
the levonorgestrel butanoate particles having a median particle size of 13 μm to 14.5 μm;
the wetting agent is polysorbate 80; and
the dispersing agent is present and is sorbitan monopalmitate.

14. The pharmaceutical formulation of claim 1, wherein:
the levonorgestrel butanoate particles having a median particle size of 13.5 μm to 14 μm;
the wetting agent is polysorbate 80; and
the dispersing agent is present and is sorbitan monopalmitate.

15. The pharmaceutical formulation of claim 1, wherein:
the levonorgestrel butanoate particles having a median particle size of 13.7 μm;
the wetting agent is polysorbate 80; and
the dispersing agent is present and is sorbitan monopalmitate.

16. The pharmaceutical formulation of claim 13, further comprising sodium carboxymethyl cellulose, sodium phosphate dibasic, sodium dihydrogen phosphate dihydrate, and benzyl alcohol.

17. The pharmaceutical formulation of claim 1, wherein the emulsifier is present and is an ionic or non-ionic surfactant.

18. A pharmaceutical formulation, comprising:
levonorgestrel butanoate particles having a median particle size ranging from 12 μm to 15 μm;
a wetting agent;
a dispersing agent, an emulsifier, or a combination thereof;
a thickening agent;
a buffer component; and
a preservative,
wherein the levonorgestrel butanoate is present at a concentration ranging from greater than 20 mg/mL to a concentration of 100 mg/mL and provided that if the pharmaceutical formulation comprises a wetting agent and a dispersing agent, then the wetting agent and the dispersing agent are present at a ratio of 3:0.1 to 5:0.1 (wetting agent:dispersing agent).

19. The pharmaceutical formulation of claim 18, wherein:
the levonorgestrel butanoate particles having a median particle size of 13 μm to 14.5 μm;
the wetting agent is polysorbate 80;
the dispersing agent is present and is sorbitan monopalmitate;
the thickening agent is sodium carboxymethyl cellulose;
the buffer component is a combination of sodium phosphate dibasic and sodium dihydrogen phosphate dihydrate; and
the preservative is benzyl alcohol.

20. The pharmaceutical formulation of claim 18, wherein the levonorgestrel butanoate particles having a median particle size of 13.5 μm to 14 μm.

21. A pharmaceutical formulation, comprising:
levonorgestrel butanoate particles having a median particle size ranging from 11 μm to 15 μm;
at least one dispersing agent;
a thickening agent;
a buffer component; and
a preservative,
wherein the levonorgestrel butanoate is present at a concentration ranging from greater than 20 mg/ml to a concentration of 100 mg/mL.

22. The pharmaceutical formulation of claim 21, wherein the levonorgestrel butanoate particles have a median particle size ranging from greater than 11.3 μm to a median particle size of 14.2 μm.

23. The pharmaceutical formulation of claim 21, wherein the dispersing agent is sorbitan monopalmitate.

24. The pharmaceutical formulation of claim 21, wherein the formulation is formulated to administer a dose of levonorgestrel butanoate that ranges from 30 mg to 90 mg.

25. A method of making the pharmaceutical formulation according to claim 1, comprising:
subjecting levonorgestrel butanoate to sterile filtration to provide a filtrate comprising sterile levonorgestrel butanoate;
producing levonorgestrel butanoate particles from the filtrate by spray drying the filtrate to control the median particle size of the levonorgestrel butanoate particles; and
combining the levonorgestrel butanoate particles with one or more of the wetting agent, the dispersing agent, the emulsifier, or the combination thereof; wherein the method does not comprise using irradiation for any sterilization.

26-27. (canceled)

28. A method of making the pharmaceutical formulation according to claim 1, comprising micronizing levonorgestrel butanoate to provide the levonorgestrel butanoate particles and combining the levonorgestrel butanoate particles with one or more of the wetting agent, the dispersing agent, the emulsifier, or the combination thereof; wherein the method does not comprise (i) using irradiation for any sterilization or (ii) using gamma or e-beam irradiation.

29. (canceled)

30. A method of providing contraception to a subject, comprising administering a dosage of levonorgestrel butanoate ranging from 20 mg to 140 mg by administering the formulation of claim 1 to the subject via injection.

31. The method of claim 30, wherein injection comprises subcutaneous injection or intramuscular injection.

32. (canceled)

33. The method of claim 30, wherein the concentration of levonorgestrel butanoate in the formulation is 70 mg/mL.

34. A method of treating or preventing a progestin-related disease or condition in a subject, comprising administering the formulation of claim 1 to the subject.
